(19) United States
(12) Patent Application Publication
(10) Pub. No.: US 2007/0191492 A1
(43) Pub. Date: Aug. 16, 2007

(54) Method for suppressing lung tumorigenesis

(75) Inventor: Reiko Tanaka, Ibaraki-shi (JP)

Correspondence Address: SUGHRUE MION, PLLC, 2100 PENNSYLVANIA AVENUE, N.W., SUITE 800, WASHINGTON, DC, 20037, US

(73) Assignee: Sumitomo Chemical Company, Limited

(21) Appl. No.: 10/562,011

(22) PCT Filed: Jun. 24, 2003
(86) PCT No.: PCT/JP03/07953

§ 371 Date: Feb. 9, 2007

(51) Int. Cl. A61K 31/09

(52) U.S. Cl. 514/715

(57) ABSTRACT

The invention provides a method of treatment for suppressing or inhibiting lung tumorigenesis, comprising administering to a subject who requires the treatment an effective amount of a compound represented by the following formula (I).

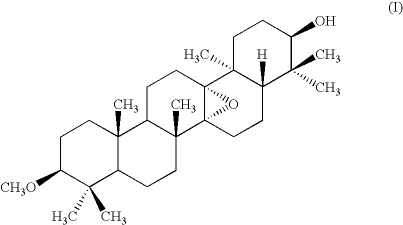

### TECHNICAL FIELD

The present invention relates to a method for suppressing or preventing lung tumorigenesis.

### BACKGROUND ART

In nearly 70% of all cases, a malignant tumor of lung has already been reached stage III or stage IV when it is detected. There are many of these cases to which surgical treatments cannot be applied. Although chemotherapy and radiation therapy are applied to a patient with an advanced stage. there are many cases wherein effectiveness of such therapies are not necessarily sufficient.

It is said that external environmental factors contribute remarkably to lung tumorigenesis. Smoking is a major cause for lung tumorigenesis. Furthermore, materials such as asbestos, arsenic, radial ray, and chromate in environments are considered to be concerned with lung tumorigenesis. With respect to people who are exposed frequently to these external environmental factors, a so-called high-risk group of lung tumor, it is important not only to make attempts of early detection and treatment, but also to take preventive measures as to lung tumor.

Under the circumstances, developments for chemoprevention of lung tumor, more specifically development of a method of treatment for suppressing or inhibiting lung tumorigenesis including administration of compounds effective for preventing lung tumor are desired earnestly.

### DISCLOSURE OF THE INVENTION

The invention has been made in view of the situation as described above and as a result of eager study, it has been found that a compound separated and purified from barks of Picea jezoensis Carr. jezoensis being a pinaceous plant has activities for suppressing or inhibiting lung tumorigenesis and is effective for preventing lung tumor, and hence, the present invention has been completed.

Namely, the invention provides:

1. a method of treatment for suppressing or inhibiting lung tumorigenesis, comprising administering to a subject who requires the treatment an effective amount of a compound represented by the following formula (I); 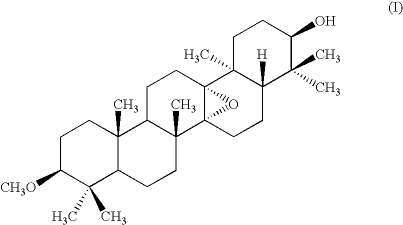
and

2. The method according to the above 1, wherein the compound is administered orally.

### BEST MODE FOR CARRYING OUT THE INVENTION

The compound represented by the above formula (I) (hereinafter, sometimes referred to as “compound 1”) can be obtained, for example, by isolating and purifying from barks, strobiles, and leaves of such as pinaceous plants, more specifically for example, barks of Picea jezoensis Carr. jezoensis and the like; or applying conventional chemical reactions to compounds isolated and purified from such natural products. The isolation and purification steps includes, for example, first immersing barks of Picea jezoensis Carr. jezoensis and the like into an organic solvent such as ethyl ether, ethyl acetate, acetonitrile, acetone, methanol, ethanol, dichloromethane, chloroform, toluene, benzene, hexane, and heptane, or a solvent prepared by admixing some of them at room temperature or a temperature of about 37° C. to about 60° C.; filtrating the immersed barks; and then distilling away the solvent from the filtrate. The resulting residue is dissolved into a suitable organic solvent such as ethyl ether, ethyl acetate, acetonitrile, acetone, methanol, ethanol, dichloromethane, chloroform, toluene, benzene, hexane, and heptane, or a solvent prepared by admixing some of them, and the resulting solution is fractionated by means of column chromatography, high performance liquid chromatography, thin-layer chromatography or the like wherein silica gel, alumina, cellulose powder or the like is used,or a combination of these methods. Specifically, it is possible to conduct the above-described purification in accordance with methods described in a literature [Tanaka, R. et al., Cancer Lett., 172, 119 (2001)] and the like.

A chemical structure of the compound 1 thus obtained may be confirmed by measuring infrared absorption spectrum, ultraviolet absorption spectrum, mass spectrum, NMR spectrum or the like of the compound 1.

Furthermore, activities involved in the compound 1 for suppressing or inhibiting lung tumorigenesis can be confirmed by, for example, such a manner that the compound 1 is administered to experimental animals to which a chemical substance inducing lung tumor has been previously administered, and a frequency of lung tumorigenesis in the experimental animals is detected. Specifically, it is possible to detect the above-described activities according to, for example, a method described in a literature [Yang, Y-M. et al., Cancer Research 62: 2-7, 2002; Yamagishi, M. et al., Cancer Letters 191(1): 49-57, 2003] and the like.

The compound 1 may be, for example, an active ingredient for an agent effective for preventing lung tumor by suppressing or preventing lung tumorigenesis, a so-called chemopreventive agent for lung tumor. The method of the present invention including administration of the compound 1 to a subject has an effect of suppressing or preventing lung tumorigenesis, and it is useful for preventing development or recurrence of lung tumor. An example of the subject includes mammals, and more specifically includes humans. The method of the invention is useful particularly for a treatment of high-risk subjects of lung tumorigenesis, for instance, subjects who are exposed frequently to external environmental factors which are known as to causal correlation with lung tumorigenesis, subjects having a personal medical history of lung tumor, subjects having a family medical history of lung tumor and the like.

The compound 1 may be administered as an active ingredient of medicines, foods, cosmetics and the like for preventing, suppressing or avoiding development or recurrence of lung tumor in the form of, for example, extracts containing the compound derived from natural products or the processed products thereof, or compositions prepared by admixing the compound with pharmaceutical carriers or excipients, food ingredients, cosmetic ingredients or the like. An amount of the compound 1 contained in these medicines, foods, cosmetics and the like is usually 0.01% by weight to 99.99% by weight. These medicines, foods, cosmetics and the like may be in the form of a variety of, for example, solids and liquids in response to their specific use applications. In addition, medicine carriers or excipients, food ingredients, cosmetic ingredients and the like which used in the compositions may be suitably selected depending on their specific applications. An example of a route of administration for the compound 1 includes oral administration, parenteral administration, and local administration, and preferable is oral administration.

In the case where the compound 1 is used as an active ingredient in medicine, a form for administration can be suitably selected as needed. A specific example of the form includes oral agents such as tablets, capsules, granules, and powdered medicines as well as parenteral agents such as parenteral injections, lotions, ointments, and suppositories, and preferable are oral agents.

Although an applied dose of oral agents varies dependent upon a variety of factors such as age and/or weight of a subject, a type of carcinogens considered to be a cause of tumorigenesis or an amount and a period of time of exposure to such a carcinogen, it is usually about 0.1 mg to about 2000 mg/day in a weight of the compound 1 for an adult. Such a daily applied dose may be divided into once or several times.

An oral agent is manufactured in accordance with a conventional method by using excipients such as lactose, starch, sucrose, glucose, mannitol, corn starch, and inorganic salts. In addition to the above-described excipients, binders, disintegrators, surfactants, emulsifiers, lubricants, humectants, plasticizers, preservatives, flavoring substances, coloring agents, fragrances and the like may be used as needed. An example of binders includes starch, dextrin, powdered acacia, hydroxypropyl starch, crystalline cellulose, ethyl cellulose, methyl cellulose, sodium carboxymethyl cellulose, hydroxypropyl cellulose, polyvinyl pyrrolidone and the like. An example of surfactants includes sodiumlauryl sulfate, soybean lecithin, egg-yolk lecithin, sucrose fatty acid esters, Polysolbate 80 and the like. An example of lubricants includes talc, waxes, hydrogenated vegetable oils, sucrose fatty acid esters, magnesium stearate, calcium stearate, aluminum stearate and the like. An example of plasticizers includes light anhydrous silicic acid, dried aluminum hydroxide gel; synthesized aluminum silicate, magnesium silicate and the like.

Liquid formulations such as suspensions, emulsions, syrups, elixirs and the like among oral agents may contain a flavoring agent, and a coloring agent.

Although an applied dose of parenteral agents varies dependent upon a variety of factors such as age and/or weight of a subject, a type of carcinogens considered to be a cause of tumorigenesis or an amount and a period of time of exposure to such a carcinogen, and manners of administration, it is usually about 0.01 mg to about 2000 mg/day in a weight of the compound 1 for an adult. Such a daily applied dose may be divided into once or several times.

A parenteral injection is manufactured in accordance with a conventional method, and to which a disinfectant, an antiseptic agent, a stabilizer and the like may be added as needed. An example of a diluent used for parenteral injections includes usually distilled water for injection, saline, glucose aqueous solution, vegetable oils for injection purposes (e.g. sesame oil, peanut oil, soybean oil, and corn oil), propylene glycol, polyethylene glycol and the like.

Examples of the other parenteral formulations include percutaneous absorbing agents such as lotions and ointments; a suppository for intrarectal administration; a respiratory tonic for administering into pulmonary airway. These parenteral. formulations are manufactured in accordance with conventional methods, respectively.

### EXAMPLES

In the following, the invention is more fully described by referring to examples, but it is to be noted that the invention is not limited thereto.

### Example 1

(Preparation of the Compound 1)

Barks of Picea jezoensis Carr. jezoensis collected in Oku-josankei, Sapporo City, Hokkaido in August 1997 were separated into the outer bark and the inner bark, 8.5 kg of the resulting outer bark were chopped up, and the chopped bark was immersed in 10 L of methylene chloride at 40° C. for 20 hours. After the resulting immersion fluid was filtered, the solvent was distilled away from the filtrate under reduced pressure to obtain 530.7 g of methylene chloride extract. Whole quantity of the extract was dissolved into chloroform, and subjected to a column chromatography on 7 kg of silica gel (Silica Gel 60 manufactured by Merck Corporation) which had been prepared with chloroform, whereby 2 L each of the eluant was fractionated while flowing chloroform. Fractions from 23 to 40 were collected to obtain a fraction A (40.8 g, 18×2 L), fractions from 41 to 50 were collected to obtain a fraction B (40.3 g, 10×2 L). In succession, 2 L each of the eluant was fractionated while flowing a mixed solvent of chloroform and ethyl acetate (chloroform: ethyl acetate=10:1), and fractions from 51 to 80 were collected to obtain a fraction C (45.3 g, 30×2 L). From the resulting fraction C, the compound 1 was separated and purified in accordance with the following scheme. Namely, the fraction C was subjected to the silica gel column chromatography to conduct first elution with chloroform, and then, elution was further made with a mixed solvent of chloroform and ethyl acetate (chloroform:ethyl acetate=10:1). A part of the resulting eluate was subjected to a thin-layer silica gel chromatography (chloroform:methanol=30:1) to isolate the compound 1.

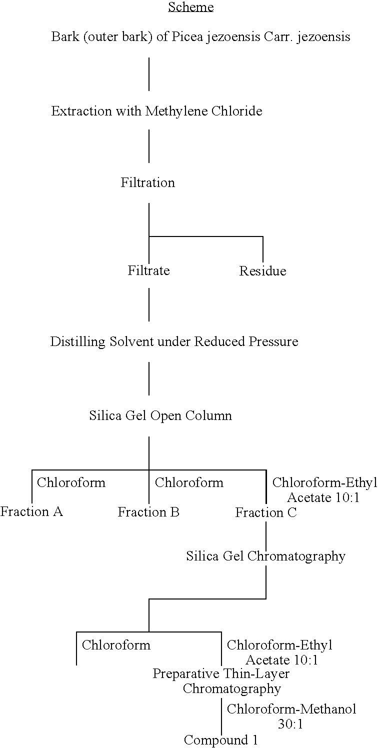

### Example 2

(Physical Property)

Physical properties of the compound 1 obtained in example 1 were measured.

### Compound 1

13α, 14α-epoxy-3β-methoxyserratan-21β-ol

Colorless needle-like crystal, C₃₁H₅₂O₃. Melting point 242-244° C. (MeOH—CHCl₃), [α]_{D}²³+31° (c 0.38, CHCl₃). Infrared spectrum IR vₘₐₓ (KBr) cm⁻¹: 3340, 2928, 2871, 1466, 1388 and 1363, 1261, 1183, 1095, 1074, 991, 975. High resolution mass spectrum M⁺m/z=472.3922 (C₃₁H₅₂O₃). NMR spectrum ¹H NMR (CDCl₃): δ 0.74 (3H, s), 0.80 (3H, s), 0.82 (3H, s), 0.92 (3H, s), 0.94 (3H, s), 1.00 (3H, s), 1.08 (3H, s), 2.52 (1H, dd, J=15.8, 8.3 Hz), 2.62 (1H, dd, J=12.2, 4.4 Hz), 3.35 (1H, t, J=2.6 Hz), 3.35 (3H, s). ¹³C NMR: δ 16.2, 16.3, 16.4, 16.8, 18.2, 21.0, 21.5. 22.2, 26.4, 27.9, 28.0, 29.3, 33.8, 36.5, 37.1, 37.9, 38.4, 38.5, 38.8, 44.8, 46.2, 54.0, 55.8, 57.5, 65.7, 72.9, 75.3, 88.3.

Mass spectrum: 472 (2) [M]⁺, 454 (8), 440 (5), 422 (8), 407 (1), 319 (4), 287 (9), 269 (14), 214 (19), 189 (20), 154 (28), 136 (100), 121 (46).

### Example 3

(Measurement of activities for suppressing lung tumorigenesis)

With respect to the compound 1 (13α, 14α-epoxy-3β-methoxyserratan-21β-ol) obtained in Example 1, activities for suppressing or inhibiting lung tumorigenesis were measured using a multi-organ carcinogenesis model in rats.

Diethylnitrosamine dissolved in saline was once administered interperitoneally to each male F344 rat at 6 weeks of age (Charles River Japan, Inc.) at a dose of 100 mg/kg body weight wherein this day was made to be day 1 of the testing. In succession, N-methyl-N-nitrosourea dissolved in saline was administered interperitoneally at a dose of 20 mg/kg body weight at day 4, 8, 11 and 15 of the testing (total four times of dosing). Furthermore, dimethylhydrazine dissolved in saline was administered subcutaneously at a dose of 40 mg/kg body weight at day 18, 22, 25, 29, 32 and 36 of the testing (total six times of dosing). In parallel with administration of these carcinogens, drinking water into which 0.05% N-butyl-N-(4-hydroxybutyl)nitrosamine was dissolved was allowed to ingest to the F344 rat from day 1 of the testing for the following four weeks. Thereafter, drinking water into which around 0.1% of 2, 2′-dihydroxy-di-n-propylnitrosamine was dissolved was allowed to ingest to the F344 rat for over two weeks from the beginning of week 5 to the end of week 6 of the testing. With respect to the F344 rat treated with a variety of carcinogens as described above, administration of the compound 1 was started from the beginning of week 8 of the testing. The compound 1 was dissolved into corn oil and forcibly administered orally into stomach using a sonde at a dose of 5 or 10 mg/kg body weight. The administration was implemented at a frequency of once a day, five days in a week, and continued for twenty three weeks. As a reference, untreated groups to which no compound 1 was administered were provided. After elapsed thirty weeks from starting the testing, each F344 rat had been sacrified to isolate a lung, and histopathological examination was conducted on the lung thus isolated. With respect to neoplastic lesions, they were classified into a benign or malignant lesion, and a number of animals each having a benign tumor or a malignant tumor and a number of tumors were counted in each group.

Test results were shown in table 1 and table 2. In either tumor, i.e. adenoma being a benign tumor or carcinoma being a malignant tumor, both indices of a frequency of animals each having tumor (table 1) and tumor multiplicity (table 2) exhibited a lower value in the group treated with the compound 1 than that of the untreated group. From these results, the compound 1 has been considered to have activities for suppressing or inhibiting development of either malignant tumor or benign tumor.

TABLE 1

| | Dosage | Frequency of Animals having Tumors ¹⁾ | | |
|---|---|---|---|---|
| Treatment | (mg/kg) | Adenoma | Carcinoma | Sum of Tumors |
| Untreated | — | 17/19 (89%) | 8/19 (42%) | 16/19 (84%) |
| Compound 1 | 5 | 11/20 (55%) | 1/20 (5%)* | 11/20 (5%)* |
| | 10 | 12/19 (67%) | 5/19 (28%) | 13/19 (72%) |

¹⁾ A number of animals observed to have tumor by histopathological examination (numerator) and a number of animals in each group (denominator) are shown in every type of tumors (adenoma or carcinoma).
*P < 0.05

TABLE 2

| | Dosage | Tumor Multiplicity¹⁾ | | |
|---|---|---|---|---|
| Treatment | (mg/kg) | Adenoma | Carcinoma | Sum of Tumors |
| Untreated | — | 1.94 ± 1.26 | 0.44 ± 0.62 | 2.26 ± 1.69 |
| Compound 1 | 5 | 0.70 ± 0.80** | 0.05 ± 0.22* | 0.75 ± 0.85** |
| | 10 | 1.00 ± 1.00* | 0.29 ± 0.47 | 1.22 ± 1.06* |

¹⁾A value obtained by dividing a whole sum of tumors in each group counted as a result of histopathological examination with a number of animals in each group (i.e. an average value of a number of tumors per an individual), and a standard deviation value are shown in every type of tumors (adenoma or carcinoma).
*P < 0.05,
**p < 0.01

### INDUSTRIAL APPLICABILITY

According to the present invention, a method of treatment for suppressing or inhibiting lung tumorigenesis can be provided.

1. A method of treatment for suppressing or inhibiting lung tumorigenesis, comprising administering to a subject who requires the treatment an effective amount of a compound represented by the following formula (I).

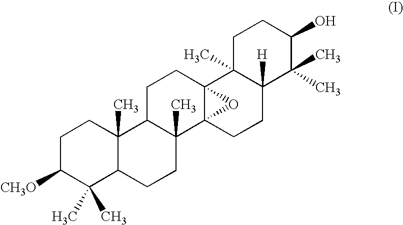

2. The method according to claim 1, wherein the compound is administered orally.
